(19) United States
(12) Patent Application Publication
(10) Pub. No.: US 2026/0225498 A1
(43) Pub. Date: Aug. 6, 2026

(54) SYSTEMS AND METHODS FOR DETECTING HEARTBEAT AND MEASURING HEART RATE

(71) Applicant: Toyota Motor Engineering & Manufacturing North America, Inc., Plano, TX (US)

(72) Inventors: Frederico Marcolino Quintao Severgnini, Ann Arbor, MI (US); Paul Donald Schmalenberg, Ann Arbor, MI (US); Bryan Pardo, Evanston, IL (US)

(21) Appl. No.: 19/042,063

(22) Filed: Jan. 31, 2025

(51) Int. Cl. B60N 2/00; A47C 17/86; A61B 5/00; A61B 5/352; B64D 11/06

(52) U.S. Cl. CPC B60N 2/0022; A47C 17/86; A61B 5/352; A61B 5/6893; A61B 5/7207; A61B 5/7264; B64D 11/06

(57) ABSTRACT

Systems and methods for detecting the heartbeat and measuring the heart rate of a human subject are disclosed herein. In one embodiment, a system receives signals from a plurality of sensors in a seat in which a human subject is sitting. The system denoises the signals using bandpass and wavelet transform filters. The system processes the denoised signals using a machine-learning-based model that performs further denoising and beamforming to detect instances of the heartbeat of the human subject and to measure the heart rate of the human subject. The machine-learning-based model is trained using ground-truth data in which an electrocardiogram waveform is replaced by Gaussian curves centered around R-peaks in the electrocardiogram waveform. The system outputs at least one of (1) one or more detected instances of the heartbeat of the human subject and (2) the measured heart rate of the human subject.

### TECHNICAL FIELD

The subject matter described herein generally relates to the analysis of time series data and, more specifically, to systems and methods for detecting the heartbeat and measuring the heart rate of a human subject.

### BACKGROUND

Recently, there has been interest in developing systems that use an acoustic sensor in a seat (e.g., a vehicle seat) to listen to heart sounds to measure the heart rate of a human subject. Such information can be valuable in assessing the level of stress or fatigue in the human subject (e.g., the driver of a vehicle) to determine whether intervention is warranted. However, the signal produced by such an acoustic sensor is very noisy, and it is difficult to extract the relevant information to estimate the subject's heart rate accurately.

### SUMMARY

An example of a system for detecting the heartbeat and measuring the heart rate of a human subject is presented herein. In one embodiment, the system comprises a seat that includes a plurality of sensors, a processor, and a memory storing machine-readable instructions that, when executed by the processor, cause the processor to receive signals from the plurality of sensors while a human subject is sitting in the seat. The memory also stores machine-readable instructions that, when executed by the processor, cause the processor to denoise the signals using a bandpass filter and a wavelet transform filter to produce denoised signals. The memory also stores machine-readable instructions that, when executed by the processor, cause the processor to process the denoised signals using a machine-learning-based model that performs further denoising and beamforming to detect instances of the heartbeat of the human subject and to measure the heart rate of the human subject. The machine-learning-based model is trained using ground-truth data in which an electrocardiogram waveform is replaced by Gaussian curves centered around R-peaks in the electrocardiogram waveform. The memory also stores machine-readable instructions that, when executed by the processor, cause the processor to output at least one of (1) one or more detected instances of the heartbeat of the human subject and (2) the measured heart rate of the human subject.

Another embodiment is a non-transitory computer-readable medium for detecting the heartbeat and measuring the heart rate of a human subject and storing instructions that, when executed by a processor, cause the processor to receive signals from a plurality of sensors in a seat in which a human subject is sitting. The instructions also cause the processor to denoise the signals using a bandpass filter and a wavelet transform filter to produce denoised signals. The instructions also cause the processor to process the denoised signals using a machine-learning-based model that performs further denoising and beamforming to detect instances of the heartbeat of the human subject and to measure the heart rate of the human subject. The machine-learning-based model is trained using ground-truth data in which an electrocardiogram waveform is replaced by Gaussian curves centered around R-peaks in the electrocardiogram waveform. The instructions also cause the processor to output at least one of (1) one or more detected instances of the heartbeat of the human subject and (2) the measured heart rate of the human subject.

Another embodiment is a method of detecting the heartbeat and measuring the heart rate of a human subject. The method includes receiving signals from a plurality of sensors in a seat in which a human subject is sitting. The method also includes denoising the signals using a bandpass filter and a wavelet transform filter to produce denoised signals. The method also includes processing the denoised signals using a machine-learning-based model that performs further denoising and beamforming to detect instances of the heartbeat of the human subject and to measure the heart rate of the human subject. The machine-learning-based model is trained using ground-truth data in which an electrocardiogram waveform is replaced by Gaussian curves centered around R-peaks in the electrocardiogram waveform. The method also includes outputting at least one of (1) one or more detected instances of the heartbeat of the human subject and (2) the measured heart rate of the human subject.

### BRIEF DESCRIPTION OF THE DRAWINGS

The accompanying drawings, which are incorporated in and constitute a part of the specification, illustrate various systems, methods, and other embodiments of the disclosure. It will be appreciated that the illustrated element boundaries (e.g., boxes, groups of boxes, or other shapes) in the figures represent one embodiment of the boundaries. In some embodiments, one element may be designed as multiple elements or multiple elements may be designed as one element. In some embodiments, an element shown as an internal component of another element may be implemented as an external component and vice versa. Furthermore, elements may not be drawn to scale.

FIG. 1 illustrates one embodiment of a vehicle within which systems and methods disclosed herein may be implemented.

FIG. 2 is a block diagram of a heartbeat detection system, in accordance with an illustrative embodiment of the invention.

FIGS. 3A and 3B illustrate a vehicle seat that includes a plurality of sensors used to detect the heartbeat of a human subject, in accordance with an illustrative embodiment of the invention.

FIG. 4 illustrates a coordinate system used in detecting and analyzing the heartbeat of a human subject, in accordance with an illustrative embodiment of the invention.

FIG. 5A is a block diagram of an architecture for a Channel-Attention Dense U-Net neural network that includes attention blocks, in accordance with the prior art.

FIG. 5B is a block diagram of a down-block in the architecture diagrammed in FIG. 5A, in accordance with the prior art.

FIG. 5C is a block diagram of an up-block in the architecture diagrammed in FIG. 5A, in accordance with the prior art.

FIG. 5D is a block diagram of a channel attention block in the architecture diagrammed in FIG. 5A, in accordance with the prior art.

FIG. 6 is a block diagram of a novel, modified architecture for a Channel-Attention Dense U-Net neural network that includes attention blocks, in accordance with an illustrative embodiment of the invention.

FIG. 7 is a graph of an electrocardiogram waveform and a waveform made up of Gaussian curves centered around the R-peaks of the electrocardiogram waveform, in accordance with an illustrative embodiment of the invention.

FIG. 8 is a flowchart of a method of detecting the heartbeat and measuring the heart rate of a human subject, in accordance with an illustrative embodiment of the invention.

To facilitate understanding, identical reference numerals have been used, wherever possible, to designate identical elements that are common to the figures. Additionally, elements of one or more embodiments may be advantageously adapted for utilization in other embodiments described herein.

### DETAILED DESCRIPTION

Various embodiments of systems and methods for detecting the heartbeat and measuring the heart rate of a seated human subject (hereinafter sometimes referred to as simply a “subject”) described herein overcome the shortcomings of the prior art by using multiple sensors and machine-learning techniques to denoise and beamform the signal to more accurately and precisely measure the heart rate of the subject. Though some of the various embodiments described herein are directed to a vehicle seat and to detecting the heartbeat and measuring the heart rate of a vehicle driver or passenger, the principles and techniques described herein can be applied to any seat or chair in which sensors can be embedded proximate to the subject's heart. Examples of other applications for the various embodiments of a heartbeat detection system described herein include, without limitation, an airplane seat, a medical chair, a medical bed, and a sofa chair.

The various embodiments described herein modify and improve upon a known architecture for multi-channel time-series analysis, a Channel-Attention Dense U-Net neural network that includes attention blocks. The various embodiments add important preprocessing techniques that denoise the input signals from a plurality of vibration sensors. More specifically, the various embodiments apply a bandpass filter and a wavelet transform filter to the input sensor signals to reduce noise and to accentuate heartbeat-related features in the input sensor signals. The various embodiments then process the preliminarily denoised sensor signals using a machine-learning-based model based on the U-net-and-attention-blocks combination just mentioned to perform further denoising and beamforming that enables the system to detect instances of the heartbeat of the human subject and to measure the heart rate of the human subject with high accuracy and precision. A further innovation in the various embodiments is that the machine-learning-based model is trained using ground-truth data in which an electrocardiogram (ECG) waveform is replaced by a waveform of Gaussian curves centered around the R-peaks in the ECG waveform. The various embodiments employ a double loss function that combines a pointwise (point-to-point) Log-Cosh loss (logarithm of the hyperbolic cosine of the prediction error) and a sum-of-squared-errors (SSE) loss function, where the latter is a comparison of the peaks only between the output of the machine-learning-based model and the ground-truth waveform made up of Gaussian curves. In some embodiments, the double loss function is the product of the Log-Cosh and the SSE loss functions. In other embodiments, the double loss function is a linear combination of the Log-Cosh and the SSE loss functions.

The heartbeat detection system outputs at least one of (1) one or more detected instances of the heartbeat of the human subject and (2) the measured heart rate of the human subject. In other words, the system outputs data pertaining to one or more detected heartbeats of the subject, the measured heart rate of the subject, or both. In some embodiments, the machine-learning-based model includes a convolutional neural network (CNN) that combines the processed (denoised and beamformed) sensor signals into a single-channel output signal.

In some embodiments, in response to one or more detected instances of the heartbeat of the human subject or the measured heart rate of the subject, the heartbeat detection system automatically takes an action to assist the human subject. The action taken varies, depending on the embodiment. In non-vehicular embodiments, the action taken can include, without limitation, notifying the human subject regarding the human subject's measured stress level; notifying the human subject of a potential medical condition (e.g., relating to heart rhythm) that requires investigation or intervention by a qualified physician; and notifying medical personnel, a family member, or friend regarding the subject's detected condition.

In a vehicular embodiment, the action taken can include, without limitation, notifying the human subject regarding the human subject's measured stress level; notifying the subject of detected fatigue and suggesting that the subject take a break from driving; and controlling, at least in part, the operation of the vehicle automatically. For example, in some embodiments, the heartbeat detection system, via an Advanced Driver-Assistance System (ADAS), can control one or more of the vehicle's steering, braking, and acceleration at least temporarily to mitigate a situation relating to the subject's detected heartbeats and/or measured heart rate. For example, in a medical emergency detected via the heartbeat detection system, the heartbeat detection system can, via the vehicle's ADAS, avoid collisions with other vehicles and/or drive the vehicle to a safe location.

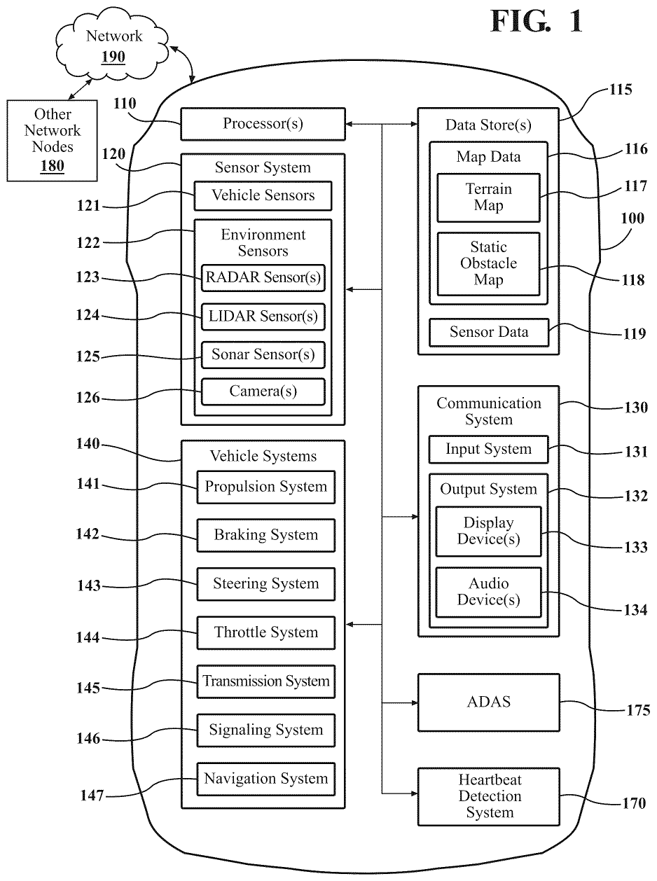

Referring to FIG. 1, an example of a vehicle 100, in which systems and methods disclosed herein can be implemented, is illustrated. The vehicle 100 can include a heartbeat detection system 170 (hereinafter sometimes referred to as simply the “system 170”) or components and/or modules thereof. As used herein, a “vehicle” is any form of motorized transport (land, water, or air). In the embodiment of FIG. 1, vehicle 100 is a land vehicle (e.g., an automobile).

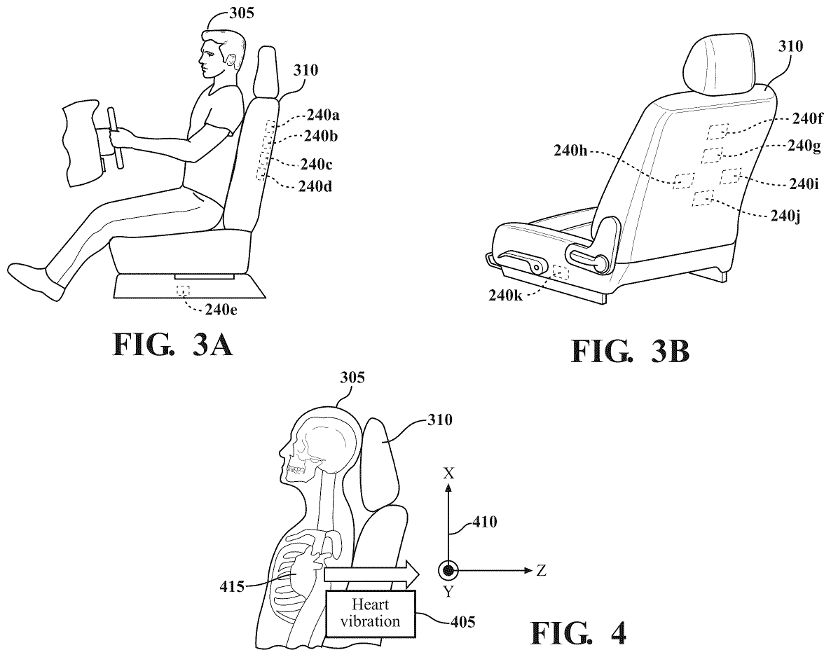

The vehicle 100 also includes various elements. It will be understood that, in various implementations, it may not be necessary for the vehicle 100 to have all the elements shown in FIG. 1. The vehicle 100 can have any combination of the various elements shown in FIG. 1. Further, the vehicle 100 can have additional elements to those shown in FIG. 1. For example, though FIG. 1 does not show seats, the vehicle 100 includes one or more seats in which the sensors of the system 170 are embedded, as discussed below in connection with FIGS. 3A and 3B. In some arrangements, the vehicle 100 may be implemented without one or more of the elements shown in FIG. 1, including the system 170. While the various elements are shown as being located within the vehicle 100 in FIG. 1, it will be understood that one or more of these elements can be located external to the vehicle 100 or be part of a system that is separate from vehicle 100. Further, the elements shown may be physically separated by large distances.

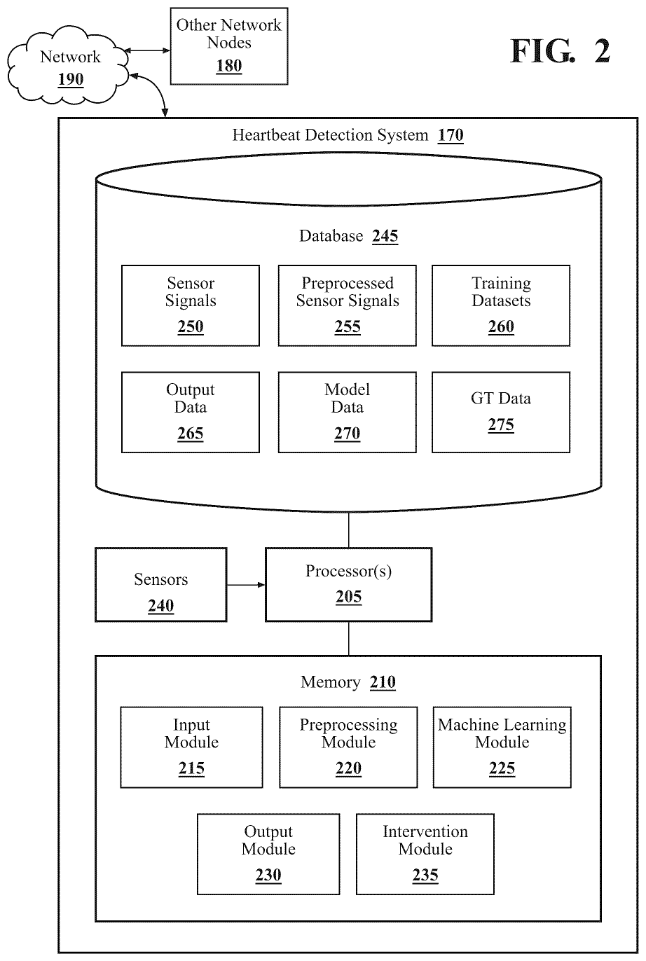

Some of the possible elements of the vehicle 100 are shown in FIG. 1 and will be mentioned in connection with subsequent figures. However, a description of many of the elements in FIG. 1 will be provided after the discussion of FIGS. 2-8 for purposes of brevity of this description. Additionally, it will be appreciated that for simplicity and clarity of illustration, where appropriate, reference numerals have been repeated among the different figures to indicate corresponding or analogous elements. In addition, the discussion outlines numerous specific details to provide a thorough understanding of the embodiments described herein. Those skilled in the art, however, will understand that the embodiments described herein may be practiced using various combinations of these elements.

Sensor system 120 can include one or more vehicle sensors 121. Vehicle sensors 121 can include one or more positioning systems such as a dead-reckoning system or a global navigation satellite system (GNSS) such as a global positioning system (GPS). Vehicle sensors 121 can also include Controller-Area-Network (CAN) sensors (sometimes herein referred to as “CAN-bus sensors”) that output, for example, speed and steering-angle data pertaining to vehicle 100. Sensor system 120 can also include one or more environment sensors 122. Environment sensors 122 generally include, without limitation, radar sensor(s) 123, Light Detection and Ranging (LIDAR) sensor(s) 124, sonar sensor(s) 125, and camera(s) 126. In some embodiments, sensor system 120 supports an ADAS 175 via which features such as park-assist, lane-change-assist, lane-keep-assist, blind-spot monitoring, adaptive cruise control, and collision avoidance can be provided.

Communication system 130 includes an input system 131 and an output system 132. The output system 132 can include components such as one or more displays 133 and one or more audio devices 134. In some embodiments, the system 170 uses the display device(s) 133 and/or the audio device(s) 134 to communicate with vehicle occupants (e.g., to issue notifications).

As shown in FIG. 1, vehicle 100 may, in some embodiments, communicate with one or more other network nodes (servers, edge servers, infrastructure devices, other connected vehicles, etc.) 180 via a network 190. In FIG. 1, network 190 represents any of a variety of wired and wireless networks. For example, in communicating directly with another vehicle, sometimes referred to as vehicle-to-vehicle (V2V) communication, vehicle 100 can employ a technology such as dedicated short-range communication (DSRC) or Bluetooth Low Energy (BLE). In communicating with a cloud or edge server or a roadside unit (RSU), vehicle 100 can use a technology such as cellular data (LTE, 5G, 6G, etc.). In some embodiments, network 190 includes the Internet.

FIG. 2 is a block diagram of a heartbeat detection system 170, in accordance with an illustrative embodiment of the invention. “Heartbeat detection system” is a shortened description of a system that detects the heartbeat of a human subject and measures (estimates) the heart rate of the human subject. As those skilled in the art are aware, a subject's heart rate is measured/estimated by counting the heartbeats within a predetermined period. For example, heart rate is typically expressed as an average in units of beats per minute (bpm). Heart rate can also be analyzed in a more granular manner by measuring fluctuations and variations in the time interval between successive heartbeats over a predetermined period.

The system 170 includes one or more processors 205. In some embodiments, the one or more processors 205 coincide partially or fully with the one or more processors 110 of vehicle 100. In such an embodiment, the system 170 may access one or more of the one or more processors 110 through a data bus or another communication path. In other embodiments, the one or more processors 205 are separate from the one or more processors 110. As shown in FIG. 2, the system 170 includes a memory 210 that stores an input module 215, a preprocessing module 220, a machine learning module 225, an output module 230, and an intervention module 235. The memory 210 is a random-access memory (RAM), read-only memory (ROM), a hard-disk drive, a flash memory, or other suitable memory for storing the modules input module 215, 220, 225, 230, and 235. The modules 215, 220, 225, 230, and 235 are, for example, computer-readable instructions that, when executed by the one or more processors 205, cause the one or more processors 205 to perform the various functions disclosed herein. In alternative arrangements, the modules 215, 220, 225, 230, and 235 are independent elements from the memory 210 that are, for example, comprised of hardware elements. Thus, the modules 215, 220, 225, 230, and 235 are alternatively ASICs, hardware-based controllers, a composition of logic gates, or other hardware-based components.

In the embodiment of FIG. 2, the system 170 includes one or more vibration sensors 240 located in one or more seats of the vehicle 100. In some embodiments, the sensors 240 are triaxial (three-axis) vibration sensors. In other embodiments, sets of three uniaxial vibration sensors with different orientations are used in place of triaxial sensors. In the embodiment of FIG. 2, a set of sensors 240 is embedded in a particular seat of the vehicle 100. The seat in question can be a driver seat or a passenger seat, depending on the embodiment. The sensors 240 are discussed in greater detail below in connection with FIGS. 3A and 3B.

As shown in FIG. 2, the system 170, in some embodiments, can communicate with one or more other network nodes (servers, edge servers, infrastructure devices, other connected vehicles) 180 via a network 190, as discussed above. Though not shown in FIG. 2, the system 170, in some embodiments, interfaces with the communication system 130 of vehicle 100, particularly display device(s) 133 and audio device(s) 134, as discussed above.

The system 170 can store various kinds of data in a database 245. In the embodiment of FIG. 2, system 170 stores sensor signals 250, preprocessed sensor signals 255, training datasets 260, output data 265, model data 270, and GT data 275. Sensor signals 250 are data (e.g., sampled waveforms) from the sensors 240. Preprocessed sensor signals 255 are data (e.g., sampled waveforms) produced by applying a bandpass filter and a wavelet transform filter to the sensor signals 250, as mentioned above. Training datasets 260 are prestored sensor signals similar to the sensor signals 250 that are used in training a machine-learning-based model that performs denoising and beamforming to enable accurate and precise detection of a subject's heartbeats and measurement of the subject's heart rate. Output data 265 includes one or more outputs (e.g., waveforms) from the machine-learning-based model and can also include information derived therefrom, such as detected instances of the subject's heartbeat and/or the subject's measured heart rate. Model data 270 includes parameters, hyperparameters, weights, etc., associated with the machine-learning-based model. Ground-truth data (GT data) 275 includes ECG waveforms corresponding to the signals in the training datasets 260 and waveforms that include Gaussian curves centered around the R-peaks in the corresponding ECG waveforms. As mentioned above, during training, the Gaussian-curves waveforms replace the original ECG waveforms to improve the training of the machine-learning-based model. These various kinds of data stored in database 245 are discussed in greater detail below.

Before summarizing the functions performed by the modules 215, 220, 225, 230, and 235, various aspects of the system 170, particularly the machine-learning-based model, will be discussed in connection with FIGS. 3A-7.

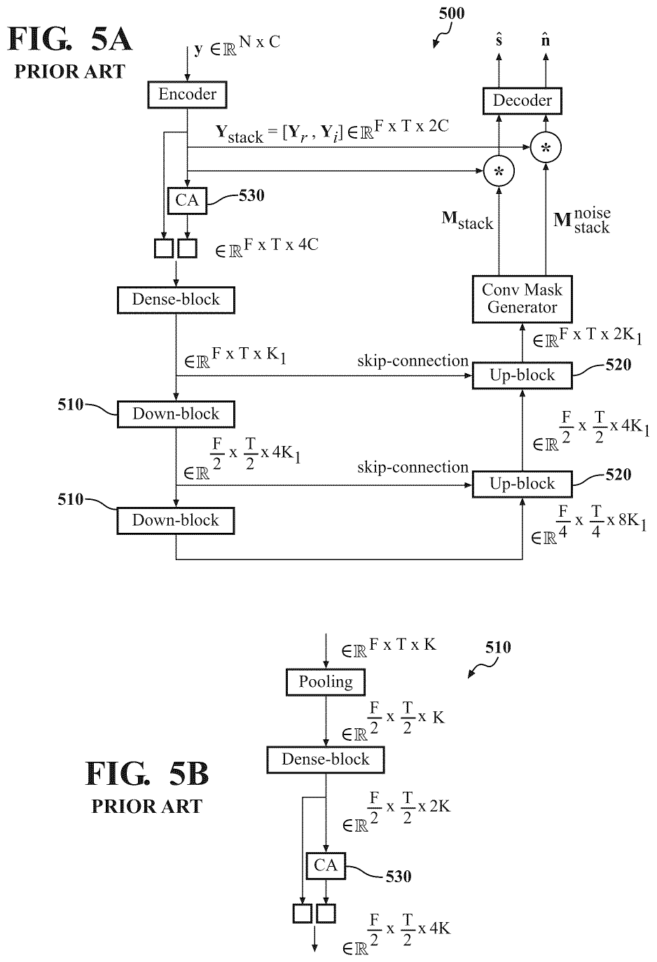

FIGS. 3A and 3B illustrate a vehicle seat that includes a plurality of sensors 240 used to detect the heartbeat of a human subject 305, in accordance with an illustrative embodiment of the invention. As shown in FIG. 3A, a plurality of sensors 240 (240a-e) have been placed (e.g., embedded) in a seat 310 occupied by a human subject 305 in a vehicle 100. In this embodiment, the sensors 240 are triaxial sensors. As mentioned above, however, each triaxial sensor can, in other embodiments, be replaced with a set of three uniaxial sensors having different orientations. In some embodiments, the sensors 240 are accelerometers that measure vibrations in the x, y, and z directions, as illustrated in FIG. 4. In the embodiment of FIG. 4, the x-axis is vertical, the z-axis is horizontal, and the y-axis is into and out of the page. In this coordinate system 410, the heart vibration 405 of the subject's (305) heart 415 is primarily in the z direction (into the back of the seat 310 where the sensors 240 are located). Therefore, a given sensor 240 picks up most of the heart vibration 405 via the z channel. The x channel and y channel pick up mostly noise, but that is useful for noise cancellation, as discussed further below. Also, it should be noted that the various sensors 240 among the plurality of sensors 240 in the seat 310 capture different signal-to-noise ratios.

In some embodiments, one or more of the sensors 240 are hydrophones, meaning microphones surrounded by a liquid such as water. The container for such a hydrophone is made of a material (e.g., acrylic plastic) that has a good acoustic impedance match with the surrounding water, which, in turn, has a good acoustic impedance match with the human body (i.e., the subject). Other fluids besides water with similar acoustic-impedance properties could also be used. Note that, in the various embodiments of the system 170, the sensors are not necessarily in direct contact with the human subject's (305) body, but this approach is not “contactless,” either.

FIG. 3B illustrates some possible locations where six sensors 240 (240f-k) can be placed in a seat 310, in accordance with an illustrative embodiment of the invention. The locations indicated in FIG. 3B are only examples, and many other configurations are possible. The objective is to place at least some of the sensors in close proximity to the subject's heart or to parts of the body where the subject's pulse is readily detectable. The sensors that are farther away from the heart pick up more noise and less heart-related signal, but, again, that is useful for noise cancellation in the subsequent processing. In the example of FIG. 3B, the heartbeat detection system 170 processes 18 channels of input sensor signals 250 (three channels for each of the six sensors 240). In other embodiments, fewer than all the possible channels output by the sensors 240 are processed. Depending on the embodiment, there can be fewer than six or more than six sensors 240. However, having a plurality of sensors (at least two sensors) is an important aspect of the various embodiments described herein because of the denoising and beamforming that it makes possible.

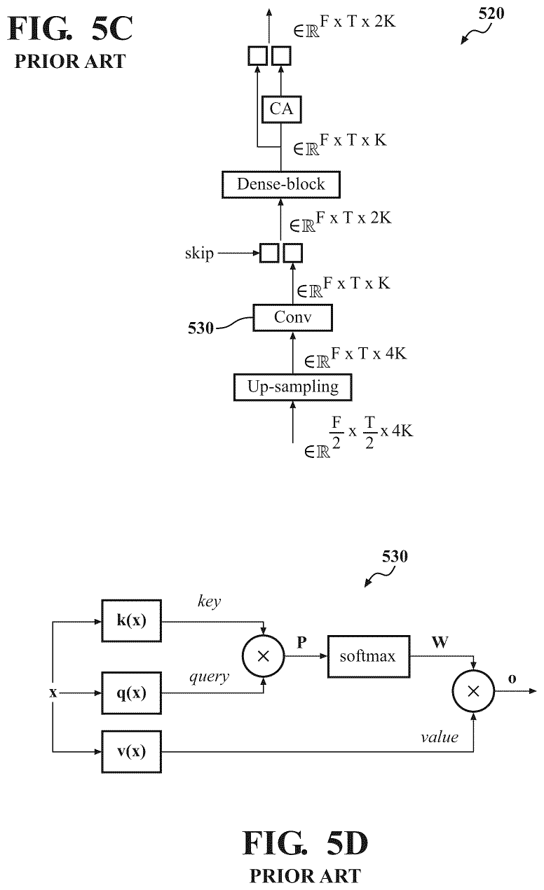

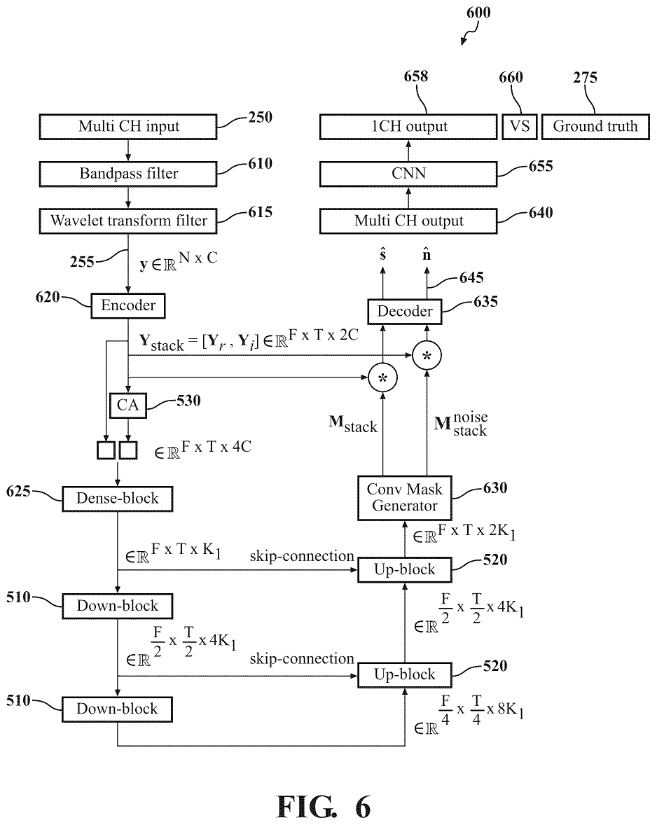

FIG. 5A is a block diagram of an architecture 500 for a Channel-Attention Dense U-Net neural network that includes attention blocks, in accordance with the prior art. The architecture 500 and the associated FIGS. 5B-5D are known in the art and are documented in B. Tolooshams et al., “Channel-Attention Dense U-Net for Multichannel Speech Enhancement,” arXiv: 2001.11542v1 [cs.SD] 30 Jan. 2020, available at https://arxiv.org/pdf/2001.11542 (Tolooshams). Tolooshams is directed to using the architecture 500 for multichannel speech enhancement, whereas the various embodiments described herein modify the architecture 500 by adding components and adjusting the operation of certain components. The modified design, discussed below in connection with FIG. 6, improves upon the architecture 500 for the purpose of detecting a subject's heartbeats and measuring a subject's heart rate. Among other components, the architecture 500 includes down-blocks 510, up-blocks 520, and a channel attention block (or simply “attention block”) 530.

FIG. 5B is a block diagram of a down-block 510 in the architecture 500 diagrammed in FIG. 5A, in accordance with the prior art. FIG. 5C is a block diagram of an up-block 520 in the architecture 500 diagrammed in FIG. 5A, in accordance with the prior art. FIG. 5D is a block diagram of a channel attention (CA) block 530 in the architecture diagrammed in FIG. 5A, in accordance with the prior art. Note that the down-blocks 510 and up-blocks 520 also include a channel attention block 530.

FIG. 6 is a block diagram of a novel, modified architecture 600 for a Channel-Attention Dense U-Net neural network that includes attention blocks, in accordance with an illustrative embodiment of the invention. Referring once again to FIG. 4, one simple example of a beamforming strategy for combining the sensor signals 250 is

$${\sum }_{n = 1}^{S} ( {CH}_{n} Z - {CH}_{n} Y - {CH}_{n} X ) ,$$

where S is the total number of sensors 240, “CHₙ” represents a particular sensor, and X, Y, or Z denotes the signal from the indicated coordinate axis of the sensor in question. In this simplified approach to beamforming, it is assumed that the heartbeat-related signal of interest is predominantly in the z-axis and that the x-axis and γ-axis of the sensor captures mostly noise, as discussed above. The machine-learning-based model shown in FIG. 6 performs a more sophisticated kind of beamforming because the model is trained to determine specific weighted combinations of the available signals from the S sensors to maximize the signal-to-noise ratio of the output. Additionally, the machine-learning-based model can learn other mathematical operations to enhance signal denoising, such as applying convolutional filters.

As shown in FIG. 6, multichannel sensor signals 250 (three from each triaxial sensor 240 in the seat 310) are input to the machine-learning-based model, as discussed above. The sensor signals 250 are preprocessed (denoised) using a bandpass filter 610 and a wavelet transform filter 615. For example, in some embodiments, the bandpass filter 610 passes frequencies from 8 to 128 Hz and attenuates frequencies outside of that band. The wavelet transform filter 615 convolves the bandpass-filtered signals in the one-dimensional (1D) domain with a wavelet resembling the basic components of a heartbeat signal/sound. This emphasizes heartbeat-related components of the signal/sound while ignoring other components. This is an additional type of denoising that is performed before the input signals from the sensors 240 reach the U-net neural network. In other words, the bandpass filter 610 and the wavelet transform filter 615 produce preprocessed sensor signals 255 that are input to the U-net. As discussed above, the machine-learning-based model including the U-net and the attention blocks perform additional denoising and beamforming to detect instances of the heartbeat of the human subject 305 and to measure the heart rate of the human subject 305.

An encoder 620 performs a Short-Time Fourier Transform (STFT). At the encoder stage, the model generates a two-dimensional (2D) representation of the preprocessed sensor signals 255 by taking the complex STFT and using both the magnitude and phase information. At the decoder stage (see decoder 635 in FIG. 6), the model converts the signals back to the time domain. That is, the 2D data is converted back to 1D time-domain data.

On the left side of FIG. 6 is a series of convolutions (dense-block 625 and down-blocks 510). On the right side of FIG. 6 is a series of transposed convolutions (up-blocks 520). This is analogous to a down-sampling process followed by an up-sampling process. The model extracts relevant features from the input signals (preprocessed sensor signals 255) of the input channels and then effectively reconstructs an output that is similar in shape to the input, except that the output includes the relevant features that support heartbeat detection and heart rate estimation.

The channel attention (CA) block 530 on the left side of FIG. 6 determines, given the presence of certain conditions, which combinations of signals from the various sensors 240 should be weighted more heavily. This permits the model to adapt to dynamically changing conditions at inference time.

As also shown in FIG. 6, the model includes a convolutional mask generator 630. In this embodiment, the model produces multichannel signal output 640 that is condensed to single-channel output data 658 by a CNN 655. In some embodiments, the multi-channel noise output 645 is not output or used. Instances of the heartbeat of the human subject 305 can be detected and the heart rate of the human subject 305 can be measured (estimated) based on the single-channel output data 658. As discussed above, the output data 265 (refer to FIG. 2) can include one or more of the single-channel output data 658, detected instances of the heartbeat of the human subject 305, and the measured heart rate of the human subject 305.

In FIG. 6, the designation “1CH output vs. ground truth” denotes that, during the training of the machine-learning-based model, the single-channel output data 658 (output waveform) is compared with GT data 275—specifically, a waveform made up of Gaussian curves at positions in time that coincide with and substitute for the R-peaks of a corresponding ground-truth ECG waveform. In FIG. 6, the element 660 represents a double loss function used in the comparison between the model's output (658) and the GT data 275. As discussed above, the double loss function 660 combines a pointwise comparison, a Log-Cosh loss, with a peaks-only comparison, which is a sum-of-squared-error (SSE) loss. That is, the SSE loss (Loss₂) is calculated in accordance with the following equation:

$${Loss}_{2} = {\sum }_{position = 0}^{K} {( {NN}_{peaks} - {GT}_{peaks} )}^{2} ,$$

where NNₚₑₐₖₛ represents the estimated peaks in the output (658) of the machine-learning-based model in FIG. 6 (“NN” stands for “neural network”), and GTₚₑₐₖₛ represents the peaks in the GT data 275 (i.e., the peaks in the ground-truth Gaussian-curves waveform mentioned above). The calculation is performed for each of K peak occurrences in the waveforms.

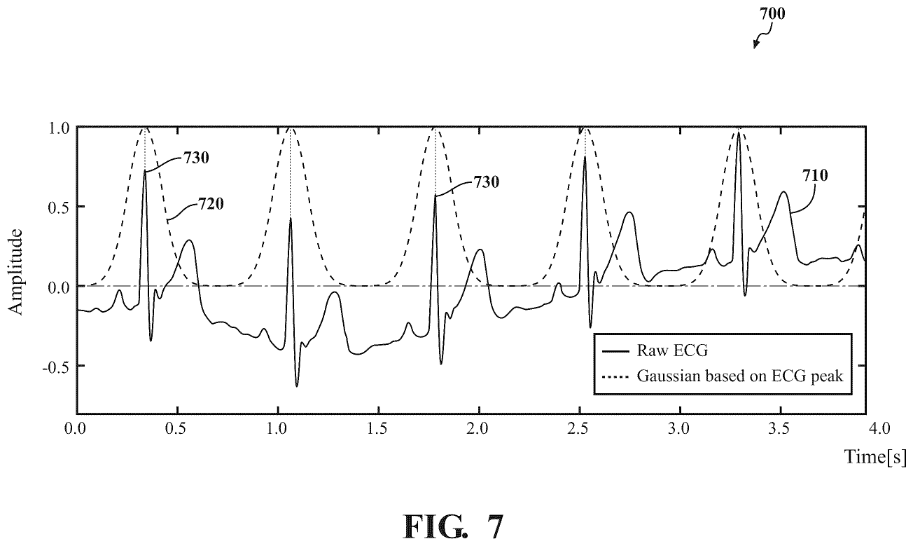

In some embodiments, the double loss function is the product of the point-by-point (Log-Cosh) and peaks-only (SSE) losses. In other embodiments, the double loss function is the weighted linear combination of the Log-Cosh loss and the SSE loss. The GT (ground-truth) data 275, particularly the Gaussian-curves waveform that replaces the ECG signal, is discussed in greater detail below in connection with FIG. 7.

FIG. 7 is a graph 700 of an ECG waveform 710 and a waveform 720 made up of Gaussian curves centered around the R-peaks 730 of the ECG waveform 710, in accordance with an illustrative embodiment of the invention. As mentioned above, both the ECG waveform 710 and the Gaussian-curves waveform 720 are types of GT (ground-truth) data 275. During the training of the machine-learning-based model, an ECG waveform 710 is obtained that corresponds with (i.e., lines up in time with) sensor signals 250 from the sensors 240. Rather than using the ECG waveform 710 itself as the ground-truth data to train the model, the various embodiments described herein instead replace the ECG waveform 710 with the waveform 720 because the waveform 720 is more stable than the counterpart ECG waveform 710 and is free of noise, which improves the quality of the training of the machine-learning-based model.

As those skilled in the art are aware, a well-known convention for labeling the parts of an ECG waveform is to divide a given heartbeat into a P wave, a QRS complex, and a T wave. The “R-peak” is the highest point of the positive deflection within the QRS complex, representing the peak electrical activity during ventricular depolarization. Two of the five R-peaks 730 in the ECG waveform 710 are labeled in FIG. 7. In generating the Gaussian-curves waveform 720, a Gaussian curve y(t) is centered around each R-peak in the ECG waveform 710 in accordance with the following equation:

$$y ( t ) = exp ( - \frac{{( t - {R}_{p t} )}^{2}}{2 {σ}^{2}} ) ,$$

where Rₚₜ is the time at which the R-peak occurs and σ² is the variance of the Gaussian curve. It is helpful to have intervals of zero between consecutive Gaussian curves, as illustrated in FIG. 7. In general, the width selected for the Gaussian curves is a function of the resting heart rate of the human subject 305. The width of the Gaussian curves can be tuned in situations in which a subject's heart rate is elevated for some reason (stress, etc.). In the embodiment of FIG. 7, all Gaussian curves in the Gaussian-curves waveform 720 are normalized to unity.

Note that the slight delay between the R-peaks of the ECG signal (an electrical phenomenon) and the heartbeat sounds whose vibrations the sensors 240 detect generally does not pose a problem because it does not affect the ability of the heartbeat detection system 170 to measure heart rate, and heart rate is the primary output of interest, in some embodiments.

Returning once again to FIG. 2, input module 215 generally includes machine-readable instructions that, when executed by the one or more processors 205, cause the one or more processors 205 to receive signals (250) from a plurality of sensors 240 in a seat 310 in which a human subject 305 is sitting. As discussed above, in some embodiments, the sensors 240 are accelerometers that measure vibrations (415) in the x, y, and z directions, as discussed above in connection with FIG. 4. In other embodiments, one or more of the sensors 240 are hydrophones. The sensors 240 and their possible placement in the seat 310 are discussed in greater detail above in connection with FIGS. 3A-4.

Preprocessing module 220 generally includes machine-readable instructions that, when executed by the one or more processors 205, cause the one or more processors 205 to denoise the signals (250) using a bandpass filter 610 and a wavelet transform filter 615 to produce denoised signals (preprocessed sensor signals 255). As discussed above, in some embodiments, the bandpass filter 610 passes frequencies from 8 to 128 Hz and attenuates frequencies outside of that band. The wavelet transform filter 615 convolves the bandpass-filtered signals in the 1D domain with a wavelet resembling the basic components of a heartbeat signal/sound. This emphasizes heartbeat-related components of the signal/sound while ignoring other components. This is an additional type of denoising that is performed before the input signals from the sensors 240 reach the Channel-Attention Dense U-Net neural network (see FIG. 6). In other words, the bandpass filter 610 and the wavelet transform filter 615 produce preprocessed sensor signals 255 that are input to the U-net.

Machine learning module 225 generally includes machine-readable instructions that, when executed by the one or more processors 205, cause the one or more processors 205 to process the denoised signals (255) using a machine-learning-based model (see architecture 600 in FIG. 6) that performs further denoising and beamforming to detect instances of the heartbeat of the human subject and to measure the heart rate of the human subject. As discussed above, the machine-learning-based model is trained using GT data 275 in which an ECG waveform 710 is replaced by Gaussian curves (720) centered around the R-peaks 730 in the ECG waveform 710.

As discussed above, at the encoder (620) stage, the model generates a 2D representation of the input signals (preprocessed sensor signals 255) by taking the complex STFT and using both the magnitude and phase information. At the decoder (635) stage, the model converts the signals back to the time domain. That is, the 2D data is converted back to 1D time-domain data.

As also discussed above, on the left side of FIG. 6 is a series of convolutions (dense-block 625 and down-blocks 510). On the right side of FIG. 6 is a series of transposed convolutions (up-blocks 520). This is analogous to a down-sampling process followed by an up-sampling process. The model extracts relevant features from the input signals (preprocessed sensor signals 255) of the input channels and then effectively reconstructs an output that is similar in shape to the input, except that the output includes the relevant features that support heartbeat detection and heart rate estimation.

As also discussed above, the channel attention (CA) block 530 on the left side of FIG. 6 determines, given the presence of certain conditions, which combinations of signals from the various sensors 240 should be weighted more heavily. This permits the model to adapt to dynamically changing conditions at inference time.

As also shown in FIG. 6, the model includes a convolutional mask generator 630. As discussed above, in this embodiment, the model produces multichannel signal output 640 that is condensed to single-channel output data 658 by a CNN 655. In some embodiments, the multi-channel noise output 645 is not output or used. Instances of the heartbeat of the human subject 305 can be detected and the heart rate of the human subject 305 can be measured (estimated) by the system 170 based on the output data 658. For example, in some embodiments, the machine-learning-based model includes one or more heads to output the heartbeat instances and the measured heart rate of the human subject 305.

Output module 230 generally includes machine-readable instructions that, when executed by the one or more processors 205, cause the one or more processors 205 to output at least one of (1) one or more detected instances of the heartbeat of the human subject and (2) the measured heart rate of the human subject 305. In other words, output module 230 outputs data pertaining to one or more detected heartbeats of the subject, the measured heart rate of the subject, or both.

Intervention module 235 generally includes machine-readable instructions that, when executed by the one or more processors 205, cause the one or more processors 205 to take automatically, in response to the one or more detected instances of the heartbeat of the human subject 305 and/or the measured heart rate of the human subject 305, an action to assist the human subject. As discussed above, the action taken varies, depending on the embodiment. In non-vehicular embodiments (e.g., a sofa chair or medical chair), the action taken can include, without limitation, notifying the human subject 305 (e.g., on a mobile device of the human subject) regarding the human subject's measured stress level; notifying the human subject 305 of a potential medical condition (e.g., relating to heart rhythm) that requires investigation or intervention by a qualified physician; and notifying medical personnel, a family member, or friend regarding the subject's detected condition.

As also discussed above, in a vehicular embodiment, the action taken can include, without limitation, notifying the human subject 305 regarding the human subject's measured stress level; notifying the subject of detected fatigue and suggesting that the subject take a break from driving; and controlling, at least in part, the operation of the vehicle 100 automatically. For example, in some embodiments, the heartbeat detection system 170, via an ADAS 175, can control one or more of the vehicle's steering, braking, and acceleration at least temporarily to mitigate a situation relating to the subject's detected heartbeats and/or measured heart rate. For example, in a medical emergency detected via the heartbeat detection system 170, the heartbeat detection system 170 can, via the vehicle's ADAS 175, avoid collisions with other vehicles and/or drive the vehicle 100 to a safe location out of the way of traffic.

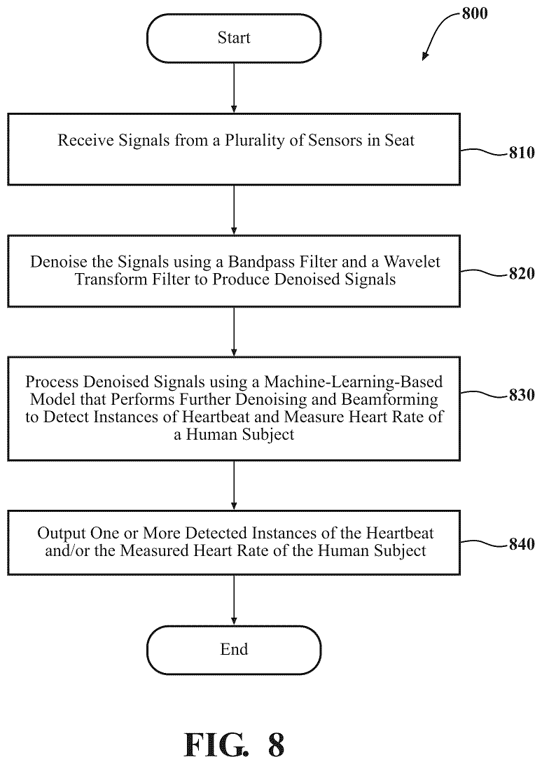

FIG. 8 is a flowchart of a method 800 detecting the heartbeat and measuring the heart rate of a human subject, in accordance with an illustrative embodiment of the invention. Method 800 will be discussed from the perspective of the heartbeat detection system 170 shown in FIG. 2. While method 800 is discussed in combination with the system 170, it should be appreciated that method 800 is not limited to being implemented within the system 170, but the system 170 is instead one example of a system that may implement method 800. As discussed above, the techniques and principles of the various embodiments described herein can be applied to other settings such as, without limitation, an airplane seat, a boat seat, a medical chair, a medical bed, and a sofa chair.

At block 810, input module 215 receives signals (250) from a plurality of sensors 240 in a seat 310 in which a human subject 305 is sitting. As discussed above, in some embodiments the sensors 240 are triaxial vibration sensors. In other embodiments, each triaxial sensor can be replaced with a set of three uniaxial sensors having different orientations. As discussed above, in some embodiments, the sensors 240 are accelerometers that measure vibrations (415) in the x, y, and z directions, as discussed above in connection with FIG. 4. In other embodiments, one or more of the sensors 240 are hydrophones. The sensors 240 and their possible placement in the seat 310 are discussed in greater detail above in connection with FIGS. 3A-4.

At block 820, preprocessing module 220 denoises the signals (250) using a bandpass filter 610 and a wavelet transform filter 615 to produce denoised signals (255). As discussed above, in some embodiments, the bandpass filter 610 passes frequencies from 8 to 128 Hz and attenuates frequencies outside of that band. The wavelet transform filter 615 convolves the bandpass-filtered signals in the 1D domain with a wavelet resembling the basic components of a heartbeat signal/sound. This emphasizes heartbeat-related components of the signal/sound while ignoring other components. This is an additional type of denoising that is performed before the input signals from the sensors 240 reach the Channel-Attention Dense U-Net neural network. In other words, the bandpass filter 610 and the wavelet transform filter 615 produce preprocessed sensor signals 255 that are input to the Channel-Attention Dense U-Net (refer to the architecture 600 in FIG. 6).

At block 830, machine learning module 225 processes the denoised signals (255) using a machine-learning-based model (see FIG. 6) that performs further denoising and beamforming to detect instances of the heartbeat of the human subject 305 and to measure the heart rate of the human subject 305. As discussed above, the machine-learning-based model is trained using GT data 275 in which an ECG waveform 710 is replaced by Gaussian curves (720) centered around R-peaks 730 in the ECG waveform 710. This is discussed in greater detail above in connection with FIG. 7.

As discussed above, at the encoder (620) stage, the model generates a 2D representation of the input signals (preprocessed sensor signals 255) by taking the complex STFT and using both the magnitude and phase information. At the decoder (635) stage, the model converts the signals back to the time domain. That is, the 2D data is converted back to 1D time-domain data.

As also discussed above, on the left side of FIG. 6 is a series of convolutions (dense-block 625 and down-blocks 510). On the right side of FIG. 6 is a series of transposed convolutions (up-blocks 520). This is analogous to a down-sampling process followed by an up-sampling process. The model extracts relevant features from the input signals (preprocessed sensor signals 255) of the input channels and then effectively reconstructs an output that is similar in shape to the input, except that the output includes the relevant features that support heartbeat detection and heart rate estimation.

As also discussed above, the channel attention (CA) block 530 on the left side of FIG. 6 determines, given the presence of certain conditions, which combinations of signals from the various sensors 240 should be weighted more heavily. This permits the model to adapt to dynamically changing conditions at inference time.

As also shown in FIG. 6, the model includes a convolutional mask generator 630. As discussed above, in this embodiment, the model produces multichannel signal output 640 that is condensed to single-channel output data 658 by a CNN 655. In some embodiments, the multi-channel noise output 645 is not output or used. Instances of the heartbeat of the human subject 305 can be detected and the heart rate of the human subject 305 can be measured (estimated) by the system 170 based on the output data 658. For example, in some embodiments, the machine-learning-based model includes one or more heads to output the heartbeat instances and the measured heart rate of the human subject 305.

At block 840, output module 230 outputs at least one of (1) one or more detected instances of the heartbeat of the human subject 305 and (2) the measured heart rate of the human subject 305. In other words, output module 230 outputs data pertaining to one or more detected heartbeats of the subject, the measured heart rate of the subject, or both.

As discussed above, in some embodiments the method 800 further includes intervention module 235 taking automatically, in response to the one or more detected instances of the heartbeat of the human subject 305 and/or the measured heart rate of the human subject 305, an action to assist the human subject. As also discussed above, the action taken varies, depending on the embodiment. In non-vehicular embodiments (e.g., a sofa chair or medical chair), the action taken can include, without limitation, notifying the human subject 305 (e.g., on a mobile device of the human subject) regarding the human subject's measured stress level; notifying the human subject 305 of a potential medical condition (e.g., relating to heart rhythm) that requires investigation or intervention by a qualified physician; and notifying medical personnel, a family member, or friend regarding the subject's detected condition.

As also discussed above, in a vehicular embodiment, the action taken can include, without limitation, notifying the human subject 305 regarding the human subject's measured stress level; notifying the subject of detected fatigue and suggesting that the subject take a break from driving; and controlling, at least in part, the operation of the vehicle 100 automatically. For example, in some embodiments, the heartbeat detection system 170, via an ADAS 175, can control one or more of the vehicle's steering, braking, and acceleration at least temporarily to mitigate a situation relating to the subject's detected heartbeats and/or measured heart rate. For example, in a medical emergency detected via the heartbeat detection system 170, the heartbeat detection system 170 can, via the vehicle's ADAS 175, avoid collisions with other vehicles and/or drive the vehicle 100 to a safe location out of the way of traffic.

FIG. 1 will now be discussed in full detail as an example vehicle environment within which the systems and methods disclosed herein may be implemented. The vehicle 100 can include one or more processors 110. In one or more arrangements, the one or more processors 110 can be a main processor of the vehicle 100. For instance, the one or more processors 110 can be an electronic control unit (ECU). The vehicle 100 can include one or more data stores 115 for storing one or more types of data. The data store(s) 115 can include volatile and/or non-volatile memory. Examples of suitable data stores 115 include RAM, flash memory, ROM, PROM (Programmable Read-Only Memory), EPROM, EEPROM (Electrically Erasable Programmable Read-Only Memory), registers, magnetic disks, optical disks, hard drives, or any other suitable storage medium, or any combination thereof. The data store(s) 115 can be a component(s) of the one or more processors 110, or the data store(s) 115 can be operatively connected to the one or more processors 110 for use thereby. The term “operatively connected,” as used throughout this description, can include direct or indirect connections, including connections without direct physical contact.

In one or more arrangements, the one or more data stores 115 can include map data 116. The map data 116 can include maps of one or more geographic areas. In some instances, the map data 116 can include information or data on roads, traffic control devices, road markings, structures, features, and/or landmarks in the one or more geographic areas. In one or more arrangement, the map data 116 can include one or more terrain maps 117. The terrain map(s) 117 can include information about the ground, terrain, roads, surfaces, and/or other features of one or more geographic areas. In one or more arrangement, the map data 116 can include one or more static obstacle maps 118. The static obstacle map(s) 118 can include information about one or more static obstacles located within one or more geographic areas.

The one or more data stores 115 can include sensor data 119. In this context, “sensor data” means any information about the sensors that a vehicle is equipped with, including the capabilities and other information about such sensors. As will be explained below, the vehicle 100 can include the sensor system 120. The sensor data 119 can relate to one or more sensors of the sensor system 120. As an example, in one or more arrangements, the sensor data 119 can include information on one or more LIDAR sensors 124 of the sensor system 120. As discussed above, in some embodiments, vehicle 100 can receive sensor data from other connected vehicles, from devices associated with other road users (ORUs), or both.

As noted above, the vehicle 100 can include the sensor system 120. The sensor system 120 can include one or more sensors. “Sensor” means any device, component and/or system that can detect, and/or sense something. The one or more sensors can be configured to detect, and/or sense in real-time. As used herein, the term “real-time” means a level of processing responsiveness that a user or system senses as sufficiently immediate for a particular process or determination to be made, or that enables the processor to keep up with some external process.

In arrangements in which the sensor system 120 includes a plurality of sensors, the sensors can function independently from each other. Alternatively, two or more of the sensors can work in combination with each other. In such a case, the two or more sensors can form a sensor network. The sensor system 120 and/or the one or more sensors can be operatively connected to the one or more processors 110, the data store(s) 115, and/or another element of the vehicle 100 (including any of the elements shown in FIG. 1).

As discussed above, the sensor system 120 can include any suitable type of sensor. Various examples of different types of sensors will be described herein. However, it will be understood that the implementations are not limited to the particular sensors described. The sensor system 120 can include one or more vehicle sensors 121. The vehicle sensors 121 can detect, determine, and/or sense information about the vehicle 100 itself, including the operational status of various vehicle components and systems.

In one or more arrangements, the vehicle sensors 121 can be configured to detect, and/or sense position and/orientation changes of the vehicle 100, such as, for example, based on inertial acceleration. In one or more arrangements, the vehicle sensors 121 can include one or more accelerometers, one or more gyroscopes, an inertial measurement unit (IMU), a dead-reckoning system, a global navigation satellite system (GNSS), a navigation system 147, and/or other suitable sensors. The vehicle sensors 121 can be configured to detect, and/or sense one or more characteristics of the vehicle 100. In one or more arrangements, the vehicle sensors 121 can include a speedometer to determine a current speed of the vehicle 100.

Alternatively, or in addition, the sensor system 120 can include one or more environment sensors 122 configured to acquire, and/or sense driving environment data. “Driving environment data” includes any data or information about the external environment in which a vehicle is located or one or more portions thereof. For example, the one or more environment sensors 122 can be configured to detect, quantify, and/or sense obstacles in at least a portion of the external environment of the vehicle 100 and/or information/data about such obstacles. The one or more environment sensors 122 can be configured to detect, measure, quantify, and/or sense other things in at least a portion the external environment of the vehicle 100, such as, for example, nearby vehicles, lane markers, signs, traffic lights, traffic signs, lane lines, crosswalks, curbs proximate the vehicle 100, off-road objects, etc.

Various examples of sensors of the sensor system 120 will be described herein. The example sensors may be part of the one or more environment sensors 122 and/or the one or more vehicle sensors 121. Moreover, the sensor system 120 can include operator sensors that function to track or otherwise monitor aspects related to the driver/operator of the vehicle 100. However, it will be understood that the implementations are not limited to the particular sensors described. As an example, in one or more arrangements, the sensor system 120 can include one or more radar sensors 123, one or more LIDAR sensors 124, one or more sonar sensors 125, and/or one or more cameras 126.

As discussed above, a vehicle 100 can further include a communication system 130. The communication system 130 can include one or more components configured to facilitate communication between the vehicle 100 and one or more communication sources. Communication sources, as used herein, refers to people or devices with which the vehicle 100 can communicate with, such as external networks, computing devices, operator or occupants of the vehicle 100, or others. As part of the communication system 130, the vehicle 100 can include an input system 131. An “input system” includes any device, component, system, element or arrangement or groups thereof that enable information/data to be entered into a machine. In one or more examples, the input system 131 can receive an input from a vehicle occupant (e.g., a driver or a passenger). The vehicle 100 can include an output system 132. An “output system” includes any device, component, or arrangement or groups thereof that enable information/data to be presented to the one or more communication sources (e.g., a person, a vehicle passenger, etc.). The communication system 130 can further include specific elements which are part of or can interact with the input system 131 or the output system 132, such as one or more display device(s) 133, and one or more audio device(s) 134 (e.g., speakers and microphones).

The vehicle 100 can include one or more vehicle systems 140. Various examples of the one or more vehicle systems 140 are shown in FIG. 1. However, the vehicle 100 can include more, fewer, or different vehicle systems. It should be appreciated that although particular vehicle systems are separately defined, each or any of the systems or portions thereof may be otherwise combined or segregated via hardware and/or software within the vehicle 100. The vehicle 100 can include a propulsion system 141, a braking system 142, a steering system 143, throttle system 144, a transmission system 145, a signaling system 146, and/or a navigation system 147. Each of these systems can include one or more devices, components, and/or combinations thereof, now known or later developed.

The one or more processors 110 can be operatively connected to communicate with the various vehicle systems 140 and/or individual components thereof. For example, returning to FIG. 1, the one or more processors 110 can be in communication to send and/or receive information from the various vehicle systems 140 to control the movement, speed, maneuvering, heading, direction, etc. of the vehicle 100. The one or more processors 110 may control some or all of these vehicle systems 140.

The vehicle 100 can include one or more modules, at least some of which are described herein. The modules can be implemented as computer-readable program code that, when executed by a processor 110, implement one or more of the various processes described herein. The processor 110 can be a device, such as a CPU, which is capable of receiving and executing one or more threads of instructions for the purpose of performing a task. One or more of the modules can be a component of the one or more processors 110, or one or more of the modules can be executed on and/or distributed among other processing systems to which the one or more processors 110 is operatively connected. The modules can include instructions (e.g., program logic) executable by one or more processors 110. Alternatively, or in addition, one or more data store 115 may contain such instructions.

In one or more arrangements, one or more of the modules described herein can include artificial or computational intelligence elements, e.g., neural network, fuzzy logic or other machine learning algorithms. Further, in one or more arrangements, one or more of the modules can be distributed among a plurality of the modules described herein. In one or more arrangements, two or more of the modules described herein can be combined into a single module.

Detailed implementations are disclosed herein. However, it is to be understood that the disclosed implementations are intended only as examples. Therefore, specific structural and functional details disclosed herein are not to be interpreted as limiting, but merely as a basis for the claims and as a representative basis for teaching one skilled in the art to variously employ the aspects herein in virtually any appropriately detailed structure. Further, the terms and phrases used herein are not intended to be limiting but rather to provide an understandable description of possible implementations. Various implementations are shown in FIGS. 1-8, but the implementations are not limited to the illustrated structure or application.

The flowcharts and block diagrams in the figures illustrate the architecture, functionality, and operation of possible implementations of systems, methods, and computer program products according to various implementations. In this regard, each block in the flowcharts or block diagrams can represent a module, segment, or portion of code, which comprises one or more executable instructions for implementing the specified logical function(s). It should also be noted that, in some alternative implementations, the functions noted in the block can occur out of the order noted in the figures. For example, two blocks shown in succession can be executed substantially concurrently, or the blocks can sometimes be executed in the reverse order, depending upon the functionality involved.

The systems, components and/or methods described above can be realized in hardware or a combination of hardware and software and can be realized in a centralized fashion in one processing system or in a distributed fashion where different elements are spread across several interconnected processing systems. Any kind of processing system or other apparatus adapted for carrying out the methods described herein is suited. A typical combination of hardware and software can be a processing system with computer-usable program code that, when being loaded and executed, controls the processing system such that it carries out the methods described herein. The systems, components and/or methods also can be embedded in a computer-readable storage, such as a computer program product or other data programs storage device, readable by a machine, tangibly embodying a program of instructions executable by the machine to perform methods and methods described herein. These elements also can be embedded in an application product which comprises all the features enabling the implementation of the methods described herein and, which when loaded in a processing system, is able to carry out these methods.

Furthermore, arrangements described herein can take the form of a computer program product embodied in one or more computer-readable media having computer-readable program code embodied or embedded, such as stored thereon. Any combination of one or more computer-readable media can be utilized. The computer-readable medium can be a computer-readable signal medium or a computer-readable storage medium. The phrase “computer-readable storage medium” means a non-transitory storage medium. A computer-readable storage medium can be, for example, but not limited to, an electronic, magnetic, optical, electromagnetic, infrared, or semiconductor system, apparatus, or device, or any suitable combination of the foregoing. More specific examples (a non-exhaustive list) of the computer-readable storage medium would include the following: an electrical connection having one or more wires, a portable computer diskette, a hard disk drive (HDD), a solid state drive (SSD), a RAM, a ROM, an EPROM or Flash memory, an optical fiber, a portable compact disc read-only memory (CD-ROM), a digital versatile disc (DVD), an optical storage device, a magnetic storage device, or any suitable combination of the foregoing. In the context of this document, a computer-readable storage medium can be any tangible medium that can contain, or store a program for use by, or in connection with, an instruction execution system, apparatus, or device.

Program code embodied on a computer-readable medium can be transmitted using any appropriate medium, including but not limited to wireless, wireline, optical fiber, cable, RF, etc., or any suitable combination of the foregoing. Computer program code for carrying out operations for aspects of the present arrangements can be written in any combination of one or more programming languages, including an object-oriented programming language such as Java™, Smalltalk, C++ or the like and conventional procedural programming languages, such as the “C” programming language or similar programming languages. The program code can execute entirely on the user's computer, partly on the user's computer, as a stand-alone software package, partly on the user's computer and partly on a remote computer, or entirely on the remote computer or server. In the latter scenario, the remote computer can be connected to the user's computer through any type of network, including a LAN or a WAN, or the connection can be made to an external computer (for example, through the Internet using an Internet Service Provider).

In the description above, certain specific details are outlined in order to provide a thorough understanding of various implementations. However, one skilled in the art will understand that the invention may be practiced without these details. In other instances, well-known structures have not been shown or described in detail to avoid unnecessarily obscuring descriptions of the implementations. Unless the context requires otherwise, throughout the specification and claims which follow, the word “comprise” and variations thereof, such as, “comprises” and “comprising” are to be construed in an open, inclusive sense, that is, as “including, but not limited to.” Further, headings provided herein are for convenience only and do not interpret the scope or meaning of the claimed invention.

Reference throughout this specification to “one or more implementations” or “an implementation” means that a particular feature, structure or characteristic described in connection with the implementation is included in at least one or more implementations. Thus, the appearances of the phrases “in one or more implementations” or “in an implementation” in various places throughout this specification are not necessarily all referring to the same implementation. Furthermore, the particular features, structures, or characteristics may be combined in any suitable manner in one or more implementations. Also, as used in this specification and the appended claims, the singular forms “a,” “an,” and “the” include plural referents unless the content clearly dictates otherwise. It should also be noted that the term “or” is generally employed in its sense including “and/or” unless the content clearly dictates otherwise.

The headings (such as “Background” and “Summary”) and sub-headings used herein are intended only for general organization of topics within the present disclosure and are not intended to limit the disclosure of the technology or any aspect thereof. The recitation of multiple implementations having stated features is not intended to exclude other implementations having additional features, or other implementations incorporating different combinations of the stated features. As used herein, the terms “comprise” and “include” and their variants are intended to be non-limiting, such that recitation of items in succession or a list is not to the exclusion of other like items that may also be useful in the devices and methods of this technology. Similarly, the terms “can” and “may” and their variants are intended to be non-limiting, such that recitation that an implementation can or may comprise certain elements or features does not exclude other implementations of the present technology that do not contain those elements or features.

The broad teachings of the present disclosure can be implemented in a variety of forms. Therefore, while this disclosure includes particular examples, the true scope of the disclosure should not be so limited since other modifications will become apparent to the skilled practitioner upon a study of the specification and the following claims. Reference herein to one aspect, or various aspects means that a particular feature, structure, or characteristic described in connection with an implementation or particular system is included in at least one or more implementations or aspect. The appearances of the phrase “in one aspect” (or variations thereof) are not necessarily referring to the same aspect or implementation. It should also be understood that the various method steps discussed herein do not have to be carried out in the same order as depicted, and not each method step is required in each aspect or implementation.

Generally, “module,” as used herein, includes routines, programs, objects, components, data structures, and so on that perform particular tasks or implement particular data types. In further aspects, a memory generally stores the noted modules. The memory associated with a module may be a buffer or cache embedded within a processor, a RAM, a ROM, a flash memory, or another suitable electronic storage medium. In still further aspects, a module as envisioned by the present disclosure is implemented as an application-specific integrated circuit (ASIC), a hardware component of a system on a chip (SoC), as a programmable logic array (PLA), or as another suitable hardware component that is embedded with a defined configuration set (e.g., instructions) for performing the disclosed functions.

The terms “a” and “an,” as used herein, are defined as one as or more than one. The term “plurality,” as used herein, is defined as two or more than two. The term “another,” as used herein, is defined as at least a second or more. The terms “including” and/or “having,” as used herein, are defined as including (i.e., open language). The phrase “at least one of . . . and . . . ” as used herein refers to and encompasses any and all possible combinations of one or more of the associated listed items. As an example, the phrase “at least one of A, B and C” includes A only, B only, C only, or any combination thereof (e.g., AB, AC, BC or ABC).

The preceding description of the implementations has been provided for purposes of illustration and description. It is not intended to be exhaustive or to limit the disclosure. Individual elements or features of a particular implementation are generally not limited to that particular implementation, but, where applicable, are interchangeable and can be used in a selected implementation, even if not specifically shown or described. The same may also be varied in many ways. Such variations should not be regarded as a departure from the disclosure, and all such modifications are intended to be included within the scope of the disclosure.

While the preceding is directed to implementations of the disclosed devices, systems, and methods, other and further implementations of the disclosed devices, systems, and methods can be devised without departing from the basic scope thereof. The scope thereof is determined by the claims that follow.

What is claimed is:

1. A system, comprising:
a seat that includes a plurality of sensors;
a processor; and
a memory storing machine-readable instructions that, when executed by the processor, cause the processor to:
receive signals from the plurality of sensors while a human subject is sitting in the seat;
denoise the signals using a bandpass filter and a wavelet transform filter to produce denoised signals;
process the denoised signals using a machine-learning-based model that performs further denoising and beamforming to detect instances of a heartbeat of the human subject and to measure a heart rate of the human subject, wherein the machine-learning-based model is trained using ground-truth data in which an electrocardiogram waveform is replaced by Gaussian curves centered around R-peaks in the electrocardiogram waveform; and
output at least one of (1) one or more detected instances of the heartbeat of the human subject and (2) the measured heart rate of the human subject.

2. The system of claim 1, wherein the machine-readable instructions include further instructions that, when executed by the processor, cause the processor to take automatically, in response to the at least one of (1) the one or more detected instances of the heartbeat of the human subject and (2) the measured heart rate of the human subject, an action to assist the human subject.

3. The system of claim 2, wherein the seat and the human subject are in a vehicle and the action to assist the human subject includes controlling, at least in part, operation of the vehicle.

4. The system of claim 1, wherein the seat is one of a vehicle seat, an airplane seat, a medical chair, a medical bed, and a sofa chair.

5. The system of claim 1, wherein the sensors include one or more of accelerometers, triaxial sensors, and hydrophones.

6. The system of claim 1, wherein the machine-learning-based model includes a neural network based on a U-Net architecture and an attention block that function together.

7. The system of claim 1, wherein the machine-learning-based model is trained using a double loss function that combines a Log-Cosh loss function and a sum-of-squared-errors loss function.

8. A non-transitory computer-readable medium storing instructions that, when executed by a processor, cause the processor to:
receive signals from a plurality of sensors in a seat in which a human subject is sitting;
denoise the signals using a bandpass filter and a wavelet transform filter to produce denoised signals;
process the denoised signals using a machine-learning-based model that performs further denoising and beamforming to detect instances of a heartbeat of the human subject and to measure a heart rate of the human subject, wherein the machine-learning-based model is trained using ground-truth data in which an electrocardiogram waveform is replaced by Gaussian curves centered around R-peaks in the electrocardiogram waveform; and
output at least one of (1) one or more detected instances of the heartbeat of the human subject and (2) the measured heart rate of the human subject.

9. The non-transitory computer-readable medium of claim 8, wherein the instructions include further instructions that, when executed by the processor, cause the processor to take automatically, in response to the at least one of (1) the one or more detected instances of the heartbeat of the human subject and (2) the measured heart rate of the human subject, an action to assist the human subject.

10. The non-transitory computer-readable medium of claim 9, wherein the seat and the human subject are in a vehicle and the action to assist the human subject includes controlling, at least in part, operation of the vehicle.

11. The non-transitory computer-readable medium of claim 8, wherein the seat is one of a vehicle seat, an airplane seat, a medical chair, a medical bed, and a sofa chair.

12. The non-transitory computer-readable medium of claim 8, wherein the machine-learning-based model includes a neural network based on a U-Net architecture and an attention block that function together.

13. The non-transitory computer-readable medium of claim 8, wherein the machine-learning-based model is trained using a double loss function that combines a Log-Cosh loss function and a sum-of-squared-errors loss function.

14. A method, comprising:
receiving signals from a plurality of sensors in a seat in which a human subject is sitting;
denoising the signals using a bandpass filter and a wavelet transform filter to produce denoised signals;
processing the denoised signals using a machine-learning-based model that performs further denoising and beamforming to detect instances of a heartbeat of the human subject and to measure a heart rate of the human subject, wherein the machine-learning-based model is trained using ground-truth data in which an electrocardiogram waveform is replaced by Gaussian curves centered around R-peaks in the electrocardiogram waveform; and
outputting at least one of (1) one or more detected instances of the heartbeat of the human subject and (2) the measured heart rate of the human subject.

15. The method of claim 14, further comprising taking automatically, in response to the at least one of (1) the one or more detected instances of the heartbeat of the human subject and (2) the measured heart rate of the human subject, an action to assist the human subject.

16. The method of claim 15, wherein the seat and the human subject are in a vehicle and the action to assist the human subject includes controlling, at least in part, operation of the vehicle.

17. The method of claim 14, wherein the seat is one of a vehicle seat, an airplane seat, a medical chair, a medical bed, and a sofa chair.

18. The method of claim 14, wherein the sensors include one or more of accelerometers, triaxial sensors, and hydrophones.

19. The method of claim 14, wherein the machine-learning-based model includes a neural network based on a U-Net architecture and an attention block that function together.

20. The method of claim 14, wherein the machine-learning-based model is trained using a double loss function that combines a Log-Cosh loss function and a sum-of-squared-errors loss function.
